(19) United States
(12) Plant Patent Application Publication
(10) Pub. No.: US 2004/0019939 P1
(43) Pub. Date: Jan. 29, 2004

(54) HYBRID CARNATION PLANT NAMED 'CFPC MEL'

(76) Inventor: Oscar T. Hasegawa, Salinas, CA (US)

Correspondence Address: CHRISTIE, PARKER & HALE, LLP, P.O. BOX 7068, PASADENA, CA, 91109-7068, US

(21) Appl. No.: 10/208,144

(22) Filed: Jul. 29, 2002

(51) Int. Cl. A01H 5/00

(52) U.S. Cl. PLT/273

(57) ABSTRACT

A new variety of carnation plant producing attractive orange picotee pink flowers.

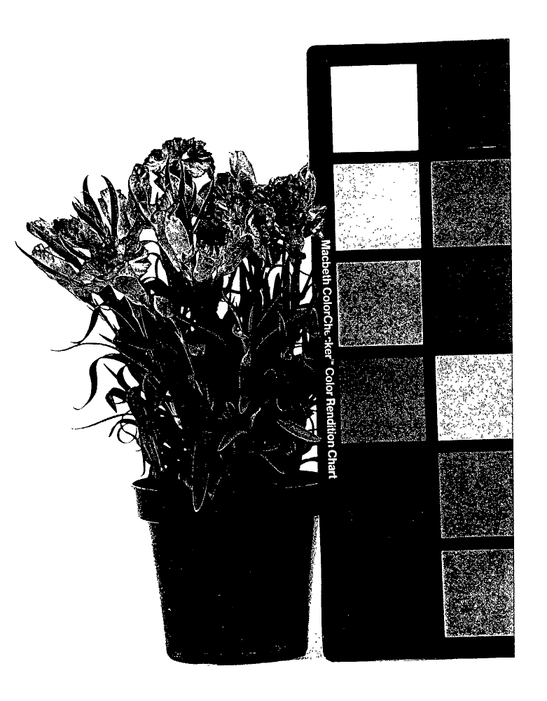

### BACKGROUND OF THE INVENTION

The present invention relates to a new and distinct variety of carnation plant which was originated is a cross of ‘CFPC MARMALADE’ (U.S. Plant Pat. No. 11,858) as a seed parent and an undistributed unpatented seedling identified as ‘970143’ as a pollen parent. The varietal denomination of the new variety is ‘CFPC MEL’.

### SUMMARY OF THE INVENTION

Among the novel characteristics possessed by the new variety which distinguish it from other varieties of which I am aware, are its attractive orange picotee pink flowers which retain color without fading. When grown in a greenhouse in Salinas, Calif., in 4½ inch plastic pots, pinched plants are about 8 inches tall and about 6 inches in diameter. Flower diameter averages about 1⅞ inches with a crown of about ¾ inch (average). Foliage is about 3⅜ inches long and about 5/16 inch wide at its widest point.

Asexual reproduction by propagation by cuttings of the new variety as performed in Salinas, Calif., shows that the foregoing and other distinguishing characteristics come true to form and are established and transmitted through succeeding propagation.

### BRIEF DESCRIPTION OF ILLUSTRATION

The accompanying illustration shows typical specimens of the vegetative growth and flowers of the new variety depicted in color as nearly true as it is reasonably possible to make the same in color illustration of this character.

### DESCRIPTION OF THE NEW VARIETY

The following is a detailed description of my new variety, with color terminology in accordance with The Royal Horticultural Society Colour Chart (RHSCC). The terminology used in color description herein refers to plate numbers in the aforementioned color chart. Phenotypical expression may vary depending upon varying environmental, seasonal and cultural conditions.

The information provided herein is based on observations of plants grown inside commercial greenhouses at Salinas, Calif. The height of plant and flower and foliage size are established as an average using standard commercial cultural practices.

Classification:

Botanical.—Hybrid Dianthus caryophyllus.

Commercial.—Carnation, standard disbud, type, suitable for cut flower production and as a pot spray.

Plant:

Height.—About 8 inches.

Form.—Herbaceous.

Growing habit.—Semi-upright.

Blooming habit.—As a spray (pot).

Blooming Season.—Year round.

Rooting time/habit.—About 18 days.

Breaking action.—Very vigorous and free-breaking plant habit with strong straight stems.

Mutation prone.—No.

Disease resistance.—Good, resistence to soil-borne diseases such as Fusarium oxysporum, and F. graminearum and root-rots such as damping off, water molds and the like as is typical of carnations.

Temperature tolerance.—Wide range.

Foilage (base of plant):

Color.—Upper side: Near 137A. Lower side: Near 137C.

Size.—About 3⅜ inches long and about 5/16 inch wide.

Quantity.—Numerous.

Shape.—Lanceolate.

Texture.—Smooth.

Ribs.—Central keel.

Arrangement.—Bi-lateral along stem.

Stem.—Color: Near 139A.

Bud.—Shape: Ovoid. Color: Near 143B. Length: About 1⅛ inches. Diameter: About 7/16 inch.

Calyx.—Sepals: About 5. Length: About 5/16 inch. Width: About 5/16 inch. Color. Outside: Near 143B. Inside: Near 145C.

Bloom:

Shape.—Rounded.

Size.—Diameter: About 1⅞ inches. Depth: About ¾ inch.

Borne.—As a spray.

Color.—General tonality: Yellow —red. Upper side: Near 20C. Lower side: Near 13D. Center: Near 20C. Outer petals: Near 55A.

Permanence.—Dry up on receptacle.

Keeping quality.—Very good.

Fragrance.—Moderate perfume.

Petalage:

Quantity.—About 30.

Outer petal size.—Length: About 1 13/16 inches. Width: About 1⅛ inches.

Central petal size.—Length: About 1⅝ inches. Width: About ⅞ inch.

Texture.—Smooth.

Appearance.—Shiny.

Form.—Fan shaped.

Reproductive Organs:

Stamen.—None.

Anthers.—None.

Filaments.—None.

Pistil.—1. Length: About ⅞ inch. Color: Whiter than 155D.

Stigma.—2. Color: Near 25D.

Fertility.—Male: Sterile. Female: Good.

Ovary.—Shape: Conical. Color. Base: Near 155D. Body: Near 144C. Apex: Near 144C.

I claim:

1. A new and distinct carnation plant of the variety substantially as herein shown and described.
